(19) United States
(12) Patent Application Publication
(10) Pub. No.: US 2012/0237482 A1
(43) Pub. Date: Sep. 20, 2012

(54) METHODS FOR TREATMENT OF NEUROLOGICAL DISORDERS BY MODULATION OF MICROGLIAL ACTIVATION

(76) Inventor: Juan Rodriguez, Lewisville, TX (US)

(21) Appl. No.: 13/423,272

(22) Filed: Mar. 18, 2012

Related U.S. Application Data
(60) Provisional application No. 61/454,241, filed on Mar. 18, 2011.

(51) Int. Cl. A61K 35/74; A61P 25/00; A61P 25/16; A61P 25/14; A61P 35/00; A61P 25/18; A61P 25/24; A61P 25/28; A61P 9/00

(52) U.S. Cl. 424/93.4

(57) ABSTRACT

Methods for treatment of neurological disorders with respect to microglial activation are presented herein. Other examples and related pharmaceutical combinations and compositions are also described herein.

### CLAIM OF PRIORITY

This is a non-provisional patent application claiming priority to U.S. Provisional Patent Application Ser. No. 61/454,241, filed on Mar. 18, 2011. The disclosure of the referenced application listed above is incorporated by reference as if recited in full herein.

### TECHNICAL FIELD

The present disclosure relates generally to medical treatments, and relates, more particularly, to methods and pharmaceutical combinations for the treatment of neurological disorders by modulation of microglial activation.

### BACKGROUND

Recently, Mario Capecchi, a distinguished professor of human genetics at the University of Utah School of Medicine and 2007 Nobel laureate in physiology and medicine showed evidence that there is a direct relationship between psychiatric disorders and the immune system, specifically cells of the immune system called microglia. (Chen et al., 2010)

A study by Johns Hopkins University School of Medicine has found evidence of microglial activation in individuals with autism (Pardo et al., 2005). Indeed, several studies now provide evidence that children with autism suffer from an ongoing neuroinflammatory process in different regions of the brain involving microglial activation (Pardo et al., 2005; Vargas et al., 2005; Zimmerman et al., 2005; Enstrom et al., 2005).

Microglia, a type of glial cell, are the resident tissue macrophage of the central nervous system (CNS). They act as the first and main form of active immune defense in the brain and spinal cord, and promote inflammation in infected or damaged tissue (Carson et al., 2007). Microglia defend the brain and spinal cord, attacking and engulfing infectious agents. Microglia search the CNS for damaged neurons, plaques, foreign bodies, and infectious agents, and can be rapidly activated by a wide range of neuropathological insults and changes (Owen and Matthews, 2011). Microglia can release a variety of cytotoxic substances, such as proteases, which when secreted by microglia catabolise specific proteins causing direct cellular damage. Cytotoxic secretion is aimed at destroying infected neurons, virus, and bacteria, however, cytotoxic secretion can also cause collateral neural damage (Gehrmann et al., 1995). For example, overactivate microglia are apt to destroy synapses between neurons (Tremblay et al., 2010), and individuals suffering from neurological disorders caused by microglial activation exhibit significant cortical synapse loss (Lue et al., 1996). In addition, once activated, microglia release large amounts nitric oxide (NO) and superoxide as a cytotoxic attack mechanism (Colton and Gilbert, 1987). Reactive oxygen and nitrogen species (ROS and RNS) derived from nitric oxide and superoxide may also cause local cellular damage by reacting with proteins, lipids, and nucleic acids (Valko et al., 2007). These chemicals can directly damage cells and lead to neuronal cell death. In addition, the release of nitric oxide at an inflammatory site may reduce and impair natural killer (NK) cell function (Takabayashi et al., 2000). Studies have found low NK function in ASD (Enstrom et al., 2009). Vojdani et al. (2008), for example, found that at least 45% of children with autism suffer from low NK cell activity.

Accordingly, a need exists for methods for treatment of neurological response disorders by modulating microglial activation.

### BRIEF DESCRIPTION OF THE DRAWINGS

The present disclosure will be better understood from a reading of the following detailed description of examples of embodiments, taken in conjunction with the accompanying figures in the drawings.

FIG. 1 presents a diagram highlighting the effect of microglial activation with respect to a disease process, using autism as an example.

FIG. 2 presents a first portion of a method for modulating microglial activation for the treatment of a neurological response disorder such as autism.

FIG. 3 presents a second portion of the method of FIG. 2.

FIG. 4 presents a third portion of the method of FIGS. 1-2.

For simplicity and clarity of illustration, the drawing figures illustrate the general manner of implementation, and descriptions and details of well-known features and techniques may be omitted to avoid unnecessarily obscuring the present disclosure. Additionally, elements in the drawing figures are not necessarily drawn to scale. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein.

### DETAILED DESCRIPTION

In one example, a method can be used for treating or ameliorating a condition with respect to a neurological disorder comprising microglial activation. The method can comprise administering to an individual suffering from the condition a probiotic dosage comprising a therapeutically effective probiotic amount, an NSAID dosage comprising a therapeutically effective NSAID amount. The NSAID dosage can be configured to modulate microglial activation in the individual. The probiotic dosage can be configured to at least one of prevent or restrict gastrointestinal lesions resulting from the NSAID dosage. The probiotic dosage can be of at least approximately 200 billion CFUs (colony-forming units) per day. In the same or other examples, the probiotic dosage can be of between approximately 200 billion CFUs to at least approximately 400 billion CFUs per day. The NSAID dosage can comprise at least one of (a) an ibuprofen dose of between approximately 30 mg/kg per day to approximately 90 mg/kg per day with respect to a weight of the individual, or (b) a dexibuprofen dose of between approximately 20 mg/kg per day to approximately 55 mg/kg per day with respect to the weight of the individual. In the same or other examples, the NSAID dosage can comprise at least one of (a) an ibuprofen dose of between approximately 60 mg/kg per day to approximately 90 mg/kg per day with respect to the weight of the individual, or (b) a dexibuprofen dose of between approximately 33 mg/kg per day to approximately 51 mg/kg per day with respect to the weight of the individual.

In one embodiment, a combination for treating or ameliorating a condition comprising a neurological disorder in an individual can comprise a probiotic dosage of a therapeutically effective probiotic amount, and an NSAID dosage of a therapeutically effective NSAID amount.

Other examples and embodiments are further disclosed herein. Such examples and embodiments may be found in the figures, in the claims, and/or in the present description.

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

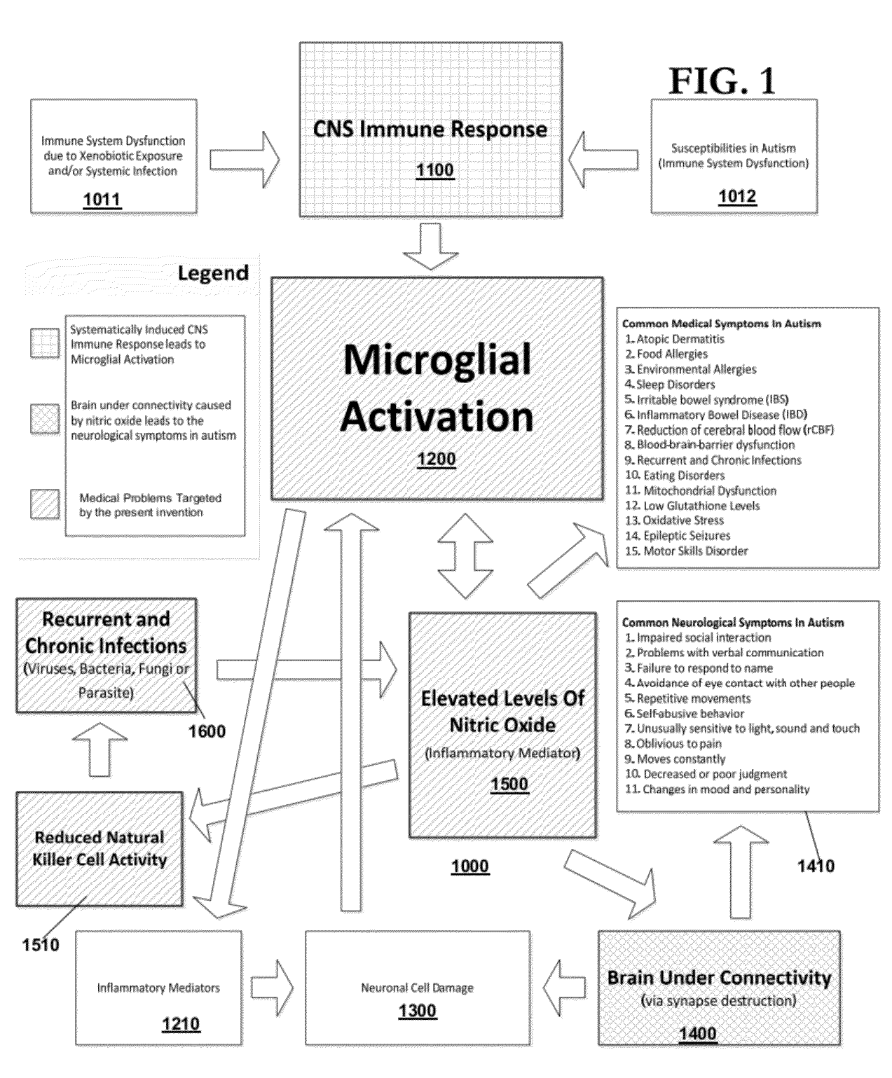

Referring now to the figures, FIG. 1 illustrates a flowchart illustrating the process of autism as a neurological disorder with respect to microglial activation. First, an immune response from the CNS is triggered (block 1100) when the individual is exposed to a xenobiotic agents or systemic infections (block 1011), and where the individual's immune system is susceptible to dysfunction (block 1012). The immune system response in block 1100 leads to microglial activation in block 1200 as a defense mechanism, but due to the immune system dysfunction (block 1012), the microglial activation is unregulated or over-extended for too long. Microglial activation (block 1200) invokes inflammatory mediators in block 1210, and over exposure to such inflammatory mediators leads to neuronal cell damage (block 1300). Microglial activation (block 1200) also causes a release of elevated levels of nitric oxide (block 1500), which can lead to brain underconnectivity (block 1400) via neuronal synapse destruction, and eventually to neuronal cell damage (block 1300). Furthermore, the elevated levels of nitric oxide (block 1500 can lead to reduced NK cell activity (block 1510), leaving the individual's immune system further weakened and susceptible to recurrent chronic infections (block 1600) such as from viruses, bacteria, fungi, or parasites. The brain underconnectivity (block 1400) caused by the different mechanisms shown in FIG. 1000 can lead to several neurological symptoms in the individual (block 1410), many of which are associated with ASD.

To control the neuroinflammation caused by undue microglial activation and nitric oxide, the use of Non-Steroidal Anti-Inflammatory Drugs (NSAIDS) has been proposed (Wood, 2003; Bendlin et al., 2010; Wilkinson et al., 2010; Richardson et al., 2005; Vandivier et al. 1999). NSAIDs, however, tend to have considerable side effects on the gastrointestinal (GI) tract, the kidneys, and the liver (Hirschowitz, 1994; Montalto et al., 2010; Ajmone-Cat et al., 2010), thus posing “critical limits to their clinical use in chronic conditions” (Ajmone-Cat et al., 2010).

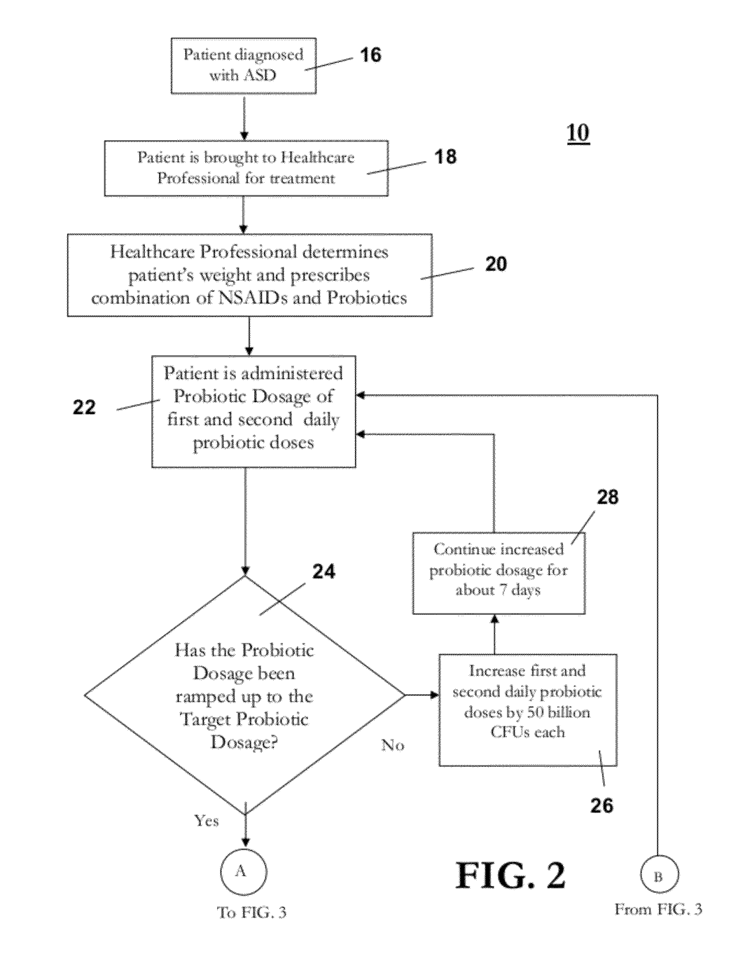

To address such shortcomings of NSAIDs, the present invention relies on a combination of NSAIDs to inhibit microglial activation, and probiotics to counteract the side effects of NSAIDS. FIG. 2 presents a flowchart of method 10 in accordance with the present disclosure to treat or ameliorate, via a combination of NSAIDs and probiotics, neurological disorder condition comprising microglial activation.

Method 10 may begin with step 16, where a patient may first be diagnosed as suffering one or more of a range of disorders associated with autism or ASD. The patient may be brought to a healthcare professional such as a doctor for treatment, as shown in step 18. At step 20, the healthcare professional may determine the patient's weight or mass, and then prescribe a combination of NSAIDs and probiotics for treatment. In some examples, the prescription can comprise an NSAID dosage based on the patient's weight or mass. In the same or other examples, the NSAID dosage may be may be compounded to a specific concentration. For instance, ibuprofen or dexibuprofen may be used in liquid concentrations of about 300 milligrams (mg) per 5 milliliters (mL) in the present or other examples.

Treatment may begin at step 22, where the patient may be administered a probiotic dosage of two probiotic doses per day. For example, a first probiotic dose may be administered in the morning on an empty stomach and at least approximately 30 minutes before the patient's first meal, and a second probiotic dose may be administered towards bed time on an empty stomach. In an exemplary embodiment, the concentration of the probiotic administered can be of at least approximately 250 billion CFUs or probiotics bacteria per gram when in powder form. In the same or other examples, the first and second probiotic doses may add up to a target probiotic dosage. There can be implementations where the target probiotic dosage can be of at least approximately 200 billion CFUs per day. In the same or other implementations, the target probiotic dosage can be of approximately 200 billion CFUs to approximately 400 billion CFU's per day. The target probiotic dosage can also be distributed between first and second target probiotic doses. For example, the first probiotic dose can be of approximately 200 billion CFU's in the morning, and the second probiotic dose can be of approximately 200 billion CFU's in the evening.

In the present example, the probiotics can be set to an initial probiotic dosage with lower CFUs to reduce the likelihood of constipation and to increase the patient's tolerance to the probiotics bacteria, and then ramped up over time to reach the target probiotic dosage per day. In some examples, the initial probiotic dose can be of approximately 100 billion CFU's per day, and may be distributed between first and second probiotic doses of approximately 50 billion CFU's per day.

For example, in step 24 of method 10, a determination may be made regarding whether the probiotics have been ramped up to the target probiotic dosage per day. If not, method 10 proceeds to step 26, where each of the first and second probiotic doses can be increased by approximately 50 billion CFUs. Method 10 then continues to step 28, where the increased first and second probiotic doses can be continued for a period of about seven days in some examples to build the patient's tolerance to the probiotics and to ensure no adverse side effects are experienced by the patient. In the same or other embodiments, the first and second doses may be increased by between 10 to 50 billion CFUs in step 26, based on the patient's response thereto. Steps 24, 26, and 28 may be repeated until the probiotics administered reach the target probiotic dosage per day.

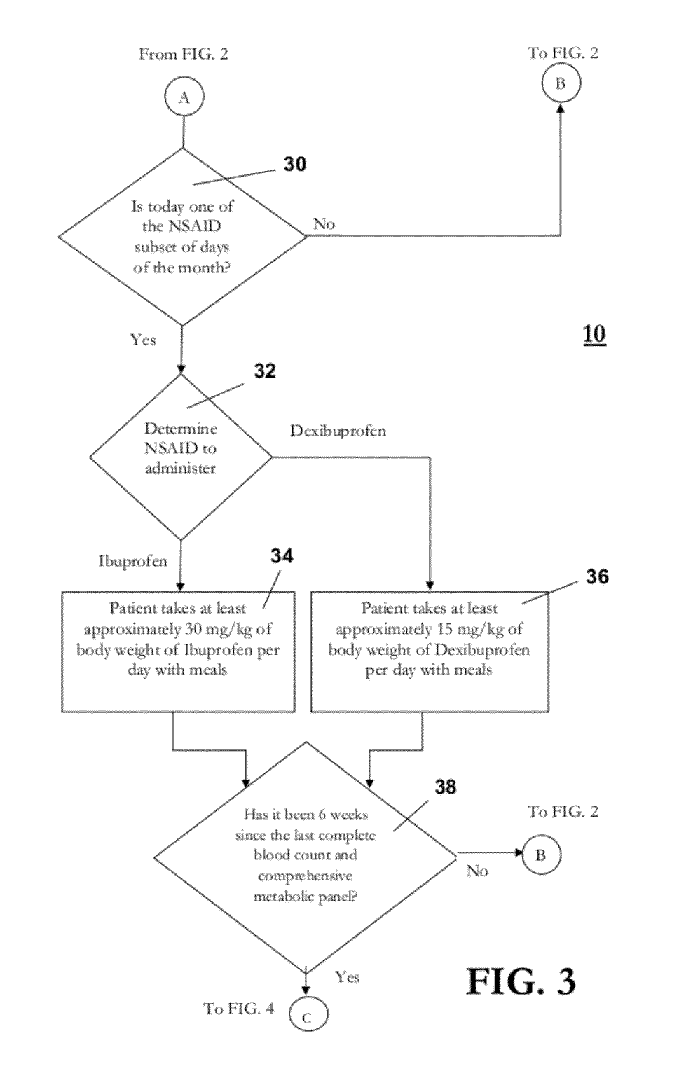

Method 10 is continued in FIG. 3 with step 30 once the target probiotic dosage per day is achieved in step 24 (FIG. 2). In the present embodiment, the NSAID dosage is configured to be administered only at a subset of days within a monthly cycle. For example, the subset of days can comprise 10 consecutive days per month. In the same or other examples, for tracking simplicity, the subset of days may be set to begin on the first day of each month. Considering the above, step 30 of method 10 can comprise determining whether the current day is within the subset of days within the month where NSAIDs will be administered. If not, method 10 returns to step 22, where the probiotic dosage is continued. Otherwise, method 10 can proceed from step 30 to step 32, where a determination can be made regarding the type of NSAID dosage to be administered.

If ibuprofen is to be administered based on the determination at step 32, method 10 can proceed to step 34, where the patient can be administered ibuprofen at a dosage of at least approximately 30 mg/kg (milligrams per kilogram or patient mass) per day with meals. For example, the ibuprofen dosage can comprise ibuprofen doses ranging from approximately 10 mg/kg to approximately 30 mg/kg three times per day with each meal. In the same or other examples the ibuprofen dosage can comprise ibuprofen doses ranging from approximately 20 mg/kg to approximately 30 mg/kg three times per day with each meal.

If dexibuprofen is to be administered based on the determination at step 32, method 10 can proceed to step 36, where the patient can be administered dexibuprofen at a dosage of at least approximately 15 mg/kg per day with meals. For example, the dexibuprofen dosage can comprise dexibuprofen doses ranging from approximately 5 mg/kg to approximately 18 mg/kg three times per day with each meal. In the same or other examples, the dexibuprofen dosage can comprise dexibuprofen doses ranging from approximately 11 mg/kg to approximately 17 mg/kg three times per day with each meal.

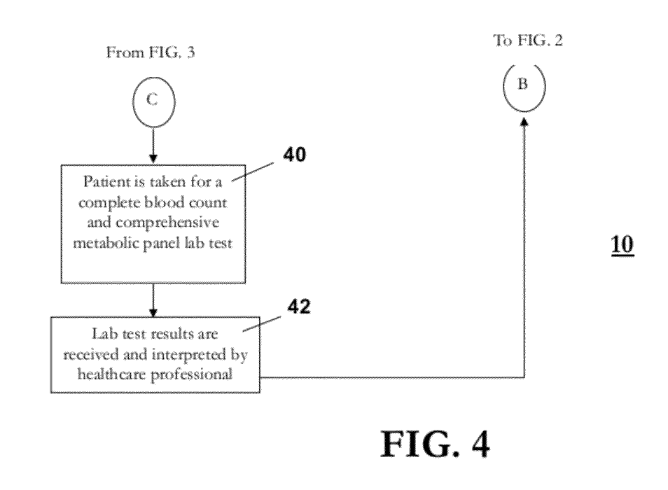

In the present embodiment, a determination may be made in step 38 regarding whether about six weeks have passed since the patient's previous blood count and comprehensive medical panel was performed. If, in step 38, it is determined that about six weeks have not passed, method 10 may continue to step 22 (FIG. 2) where the probiotic dosage is continued. If it is determined in step 38 that about six weeks have passed, method 10 can continue to step 40 in FIG. 4, where the patient may be taken for a complete blood count and comprehensive medical panel lab test. Results of the lab test may then be received or interpreted by a medical professional in step 42 to monitor the progress of the treatment.

As described above, the daily NSAID dosage may be repeated for a maximum of about ten days to avoid development of undesired gastrointestinal, kidney, or liver side effects associated therewith. The probiotics should continue to be administered on a daily basis even on days where the NSAID dosage is not administered to ensure that the patient's digestive system remains healthy. In some implementations, the probiotics used for the probiotic dosage can comprise the “D-Lactate Free Probiotic” formulation, available from Custom Probiotics of Glendale, Calif.

In the same or other embodiments, a method of treating or ameliorating a neurological disorder comprising microglial activation can comprise administering to an individual suffering from the condition a probiotic dosage comprising a therapeutically effective probiotic amount, and an NSAID dosage comprising a therapeutically effective NSAID amount. The NSAID dosage can be configured to inhibit microglial activation in the individual, and the probiotic dosage can be configured to prevent and/or restrict gastrointestinal, kidney, and/or liver lesions resulting from the NSAID dose.

There can be examples where the neurological disorder condition may comprise an autism spectrum disorder condition. In the same or other examples, the neurological disorder may comprise an Alzheimer's condition, an amyotrophic lateral sclerosis (ALS) condition, a Parkinson's disease condition, a cardiovascular disease condition, a stroke condition, a brain cancer condition, a schizophrenia condition, a depression condition, an obsessive-compulsive disorder (OCD) condition, a Huntington's disease condition, a sleep disorders condition, a Rett's syndrome condition, an adrenoleukodystrophy condition, a Tourette's syndrome condition, and/or other neurological disorder(s) comprising microglial activation.

In some embodiments, the NSAID dosage can be correlated to a weight of the individual, and there can also be embodiments where the probiotic dosage may also be correlated to the weight of the individual. The probiotic dosage can comprise at least approximately 200 billion CFUs per day in some implementations. There can also be examples where the probiotic dosage can be of between approximately 200 billion CFUs and 400 billion CFUs per day. In the same or other embodiments, the NSAID dosage can be configured to modulate microglial release of neuro-inflammatory nitric oxide. There can be embodiments where the NSAID dosage can comprise an ibuprofen dose as described above with respect to step 34 of method 10 in FIG. 3. In other embodiments, the NSAID dosage can comprise a dexibuprofen dose as described above with respect to step 36 of method 10 in FIG. 3. Other embodiments may use NSAIDs other than ibuprofen or dexibuprofen with corresponding dosages based on the weight of the individual.

The probiotic dosage and the NSAID dosage can be administered as part of a monthly treatment regimen comprising approximately 30 day cycles. In such implementations, the probiotic dosage can be configured to be administered daily throughout the monthly treatment regimen, and the NSAID dosage can be configured to be administered daily throughout a subset of days of the monthly treatment regimen. The subset of days for administering the NSAID dosage can comprise at least approximately 10 consecutive days in some examples.

There can be some implementations where the probiotic dosage can be administered in parts. For example, a first probiotic dose can be administered in the morning, and a second probiotic dose can be administered at night, where each of the first and second probiotic doses can comprises approximately half of the probiotic dosage. In the same or other implementations, the first probiotic dose can be administered at least approximately 30 minutes prior to a first daily meal of the individual, and the second probiotic dose can be administered at least approximately 30 minutes after a last daily meal of the individual.

In the same or other examples, the NSAID dosage may also be administered in parts. For example, a first NSAID dose can be administered in the morning, a second NSAID dose can be administered at around mid-day, and a third NSAID dose can be administered at night, where each of the first, second, and third NSAID doses can comprise approximately ⅓ of the NSAID dosage. The first NSAID dose may be administered along with a first daily meal of the individual, the second daily NSAID dose may be administered along with a second daily meal of the individual, and the third daily NSAID dose may be administered along with a third daily meal of the individual in some implementations.

There can also be implementations where other medications may be administered to the individual in addition to the combination of NSAIDs and probiotics. For example, an antiviral dosage comprising a therapeutically effective amount of antiviral agents may be administered to an individual against one or more microglial-activation-inducing viruses. In such examples, the antiviral dosage can comprise a valacyclovir dosage of at least approximately 21.45 mg/lb (milligrams per pound) per day, a famciclovir dosage of at least approximately 7.16 mg/lb per day, and/or an acyclovir dosage of at least approximately 36 mg/lb per day. The valacyclovir dosage can comprise in some examples an adult valacyclovir dosage of at least approximately 1 gram three times per day, or a child valacyclovir dosage of at least approximately 7.15 mg three times per day. The famciclovir dosage can comprise in some examples an adult famciclovir dosage of at least approximately 500 mg three times per day, or a child famciclovir dosage of at least approximately 3.58 mg two times per day. The acyclovir dosage can comprise in some examples an acyclovir dosage of at least approximately 9.09 mg/lb four times per day, or an acyclovir dosage of at least approximately 9.09 mg/lb five times per day.

In the same or other examples, an antifungal dosage comprising a therapeutically effective amount of antifungal agents may be administered to the individual against one or more microglial-activation-inducing fungal infections. For instance, the antifungal dosage can comprise a ketoconazole dosage of at least approximately 4 mg/kg, up to 200 mg per day, or a fluconazole dosage of at least approximately 4 mg/kg, up to 200 mg per day.

An immunomodulator dosage comprising a therapeutically effective amount of immunomodulator agents may also be administered to the individual to modulate cell-mediated immunity via T-lymphocytes and NK cell cytotoxicity regulation. If the individual weighs up to 20 kg, the immunomodulator dosage can comprises a child inosine pranobex dosage of at least approximately 50 mg/kg in some implementations. Otherwise, if the individual weighs over 20 kg, the immunomodulator dosage can comprises in some examples an adult inosine pranobex dosage of at least approximately 50 mg/kg per day (up to 3 grams), an adult inosine pranobex dosage of at least approximately 1 gram three times per day, or an adult inosine pranobex dosage of at least approximately 1 gram four times per day.

A glutathione dosage comprising a therapeutically effective amount of glutathione may also be administered to the individual to reduce oxidative stress in the brain caused by elevated levels of nitric oxide released by chronically active microglia. The glutathione dosage can be of at least approximately 250 mg per day in some examples.

An LDN (low-dose naltrexone) dosage comprising a therapeutically effective amount of naltrexone may also be administered to the individual to increase NK cell activity for fighting microglia-activating infections. In some examples, the LDN dosage can comprise a low-dose-naltrexone (LDN) dose of at least approximately 3 mg per day.

In some examples, one or more of the different steps of the methods described above may be combined into a single step or performed simultaneously, and/or the sequence of such steps can be changed. In the same or other examples, some of the steps of the methods described above may be subdivided into several sub-steps. There can also be examples where the methods described above may comprise further or different steps. Other variations can be implemented with respect to the methods described herein without departing from the scope of the present disclosure.

As described above, NSAID dosage and the probiotic dosage of the combination described above can be administered separately to the individual. For example, the probiotic dosage can be administered to the individual on an empty stomach, and the NSAID dosage can be administered along with a meal. In some implementations, the NSAID dosage and the probiotic dosage may be produced, sold, or marketed together, such as in a single package, and/or with instructions regarding how to administer the probiotic dosage and the NSAID dosage relative to each other.

There can be other embodiments, however, where a pharmaceutical composition may comprise a portion of the NSAID dosage and a portion of the Probiotic dosage. For example, the pharmaceutical composition can comprise at least approximately 25% of the therapeutically effective probiotic amount of the probiotic dosage per day, and at least approximately 25% of the therapeutically effective NSAID amount of the NSAID dosage per day, where the pharmaceutical composition may be administered to the individual three to four times per day. In the same or other examples, the pharmaceutical composition may also comprise a pharmaceutically acceptable carrier vehicle configured to disperse the portion of the probiotic dosage and the portion of the NSAID dosage in the pharmaceutical composition within the individual. There can be examples where the pharmaceutical composition and/or the pharmaceutically acceptable carrier may be in a solid or pill form, and examples where the pharmaceutical composition and/or the pharmaceutically acceptable carrier may be in a liquid solution form.

In the same or other examples, the NSAID dosage and probiotic dosage may also be administered in combination with one or more of the other medication dosages described above, such as the antiviral dosage, the antifungal dosage, the immunomodulator dosage, the glutathione dosage, and/or the LDN dosage.

In some examples, one or more of the different steps of method 10 (FIGS. 2-4) can be combined into a single steps or performed simultaneously, and/or the sequence of such steps can be changed. There can also be examples where method 10 may comprise further or different blocks. In addition, there may be examples where method 10 may comprise only part of the steps described above. Other variations can be implemented for method 10 without departing from the scope of the present disclosure.

With respect to the present disclosure, an “effective amount” is an amount sufficient to effect beneficial or desired clinical results as described herein. In terms of treatment of a mammal, e.g., a human patient, an effective amount of NSAIDs is an amount sufficient to treat, manage, palliate, ameliorate, or stabilize a condition, such as microglial activation or activity in the brain of the mammal. An effective amount can be administered in one or more doses, and may be generally determined by a physician on a case-by-case basis. Several factors are typically taken into account when determining an appropriate dosage. These factors can include age, sex and weight of the patient, the condition being treated, the severity of the condition and the form of the medication being administered.

Effective dosage forms, modes of administration, and dosage amounts may be determined empirically, and making such determinations is within the skill of the art. It is understood by those skilled in the art that dosage amount may vary depending on the route of administration, the rate of excretion, the duration of the treatment, the identity of any other drugs being administered, the age, size, and/or weight of the patient, and like factors known in the art. In general, a suitable dose of an NSAID according to the invention will be that amount of NSAID comprising the lowest dose effective to produce the desired effect. The effective dose of the NSAID may be administered as two, three, four, five, six or more sub-doses, administered separately at appropriate intervals throughout the day.

The pharmaceutical compositions and/or combinations disclosed herein may be administered in any desired and effective manner. Preferably, the administration can be oral, whether in liquid solution of solid form.

In some examples, the NSAIDs, the probiotics, and/or other medications described herein may be administered as a pharmaceutical formulation (composition). Pharmaceutically acceptable compositions of the invention comprise one or more NSAIDs and/or probiotics in admixture with one or more pharmaceutically-acceptable carriers and, optionally, one or more other compounds, drugs, ingredients and/or materials. Regardless of the route of administration selected, the NSAIDs and/or probiotics of the present disclosure can be invention are formulated into pharmaceutically-acceptable dosage forms by conventional methods known to those of skill in the art. See, e.g., Remington's Pharmaceutical Sciences (Mack Publishing Co., Easton, Pa.).

Pharmaceutically acceptable carriers are well known in the art (see, e.g., Remington's Pharmaceutical Sciences (Mack Publishing Co., Easton, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The pharmaceutical compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups, and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

### EXAMPLES

The following example is provided to further illustrate the methods and compositions of the present invention. This example is illustrative only and not intended to limit the scope of the invention in any way.

Subject Boy was born in the summer of 2004, and developed normally until reaching approximately 18 months of age (the “regression time”). The Boy was attentive, displayed full eye contact, responded to his name, and had developed vocabulary at a normal pace just prior to the regression time. The Boy developed autistic symptoms after the regression time, such as ceasing to respond to his name and losing the skills he had attained so far as his neurological health progressively deteriorated. At or about the same time of the regression time, the Boy also developed other medical problems including Vitiligo. At the age of 2, after an evaluation, a therapist from the Early Child Intervention Program advised the parents that the boy had red flags for autism. Following a viral and bacterial infection at or about 30 months of age, the Boy's autistic symptoms became severe. The Boy was officially diagnosed with autism by a developmental pediatrician at the age of 3, and qualified for the Preschool Program for Children With Disabilities (PPCD). While in the PPCD program, the Boy was evaluated by a speech therapist who diagnosed him with severe speech impairments. A video of the Boy showing severe autistic symptoms is available at http://www.youtube.com/watch?v=GbEeNJgaIQQ.

When the Boy turned 4 years old he developed another viral infection and developed a high fever that lasted over 7 days. Parents were able to keep the fever down by giving the Boy ibuprofen for children, and noticed that even though the Boy was still very ill from the viral infection, the Boy's autistic symptoms decreased while under Ibuprofen. He was more aware, calm and was saying more words. The ibuprofen was administered at about 30-40 mg/kg throughout the 7 days for the duration of the fever, but because the Boy was ill, he did not eat much during that period. Gastrointestinal problems caused by the ibuprofen soon surfaced, however. As the Parents found out when they took the Boy to the hospital to address the now-evident gastrointestinal problems, ibuprofen can have detrimental gastrointestinal side effects, and these can be worsened or accelerated by lack of food intake. The Boy was diagnosed and treated for gastrointestinal lesions and ulcers caused by the ibuprofen and the lack of food.

Shortly thereafter, a brain single-photon emission computerized tomography (SPECT) scan was performed on the Boy to measure brain function and blood flow. The SPECT scan revealed the Boy was suffering from brain inflammation, which could be contributing to his autistic symptoms. After learning of the Boy's brain inflammation, Applicant recalled the experience regarding the Ibuprofens' effect on decreasing the Boy's autistic symptoms. Upon further research, Applicant learned of the role or microglial activation as a response to infections and with respect to brain inflammation as part of the brain's immune system, and of the ability of NSAIDS for controlling microglial activation. Applicant then began administering NSAID dosages to the Boy to modulate microglial activation and reduce brain inflammation, using ibuprofen dosages of approximately 30 mg/kg to approximately 90 mg/kg per day with meals. Applicant also arrived at a dosage of 200-400 Billion CFUs of probiotics per day, and administered such dosage to the Boy to address and prevent the gastrointestinal side effects of ibuprofen mentioned above by replenishing the beneficial bacteria in the digestive tract and stomach lining. Meanwhile, treatment continued for the Boy's chronic viral, bacterial and fungal infections, including human herpesvirus 6 (HHV-6) infections, clostridium difficile bacterial infections, and yeast infections.

Through treatment with the NSAIDs and probiotics, the boy's autistic symptoms waned and eventually disappeared. The treatment lasted approximately three years, and the boy improved from being a severely autistic child to being perfectly normal and healthy. A video presenting the Boy devoid of autistic symptoms after the NSAID treatment is available at http://www.youtube.com/watch?v=QKvcOOi68Ns. The boy no longer needs to take any medications to stay healthy physically and mentally. Food allergies, environmental allergies, recurrent bacterial, yeast, fungal and viral infections are no longer a problem. The Vitiligo was completely cured and the neurological symptoms he suffered from disappeared. The boy graduated from speech therapy and applied behavioral analysis (ABA) therapy. The boy's speech is up to par with his peers, and was substantially non-existent prior to the NSAID treatment. Motor skills issues are also gone, where they Boy now has very good muscle tone and can play sports which were a very big challenge physically for him just a few years ago. The boy is also very social now. He likes to talk and have conversations with his friends and family, he likes having his friends over at our house, he plays the piano, loves school and has very good grades, and is also popular amongst the children in his school. He also has a great sense of humor. In summary, the boy went from being a child that was completely removed from his surroundings to a child that is very alert, social, and smart.

Although the methods for treatment of neurological disorders by modulation of microglial activation and related pharmaceutical combinations and compositions herein have been described with reference to specific embodiments, various changes may be made without departing from the spirit or scope of the present disclosure. Additional examples of such changes and others have been given in the foregoing description. Other permutations of the different embodiments having one or more of the features of the various figures are likewise contemplated. Accordingly, the specification, claims, and drawings herein are intended to be illustrative of the scope of the disclosure and is not intended to be limiting. It is intended that the scope of this application shall be limited only to the extent required by the appended claims.

The methods for treatment of neurological disorders by modulation of microglial activation and related pharmaceutical combinations and compositions discussed herein may be implemented in a variety of embodiments, and the foregoing discussion of certain of these embodiments does not necessarily represent a complete description of all possible embodiments. Rather, the detailed description of the drawings, and the drawings themselves, disclose at least one preferred embodiment, and may disclose alternative embodiments.

All elements claimed in any particular claim are essential to the embodiment claimed in that particular claim. Consequently, replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are expressly stated in such claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### DOCUMENTS

All documents cited are incorporated by reference as if recited in full herein.
Ajmone-Cat, Maria Antonietta; Bernardo, Antonietta; Greco, Anita; Minghetti, Luisa. 2010. Non-Steroidal Anti-Inflammatory Drugs and Brain Inflammation: Effects on Microglial Functions. Pharmaceuticals 3, no. 6: 1949-1965.
Bendlin, B. B., Newman, L. M., Ries, M. L., Puglielli, L., Carlsson, C. M., Sager, M. A., Rowley, H. A., Gallagher, C. L., Willette, A. A., Alexander, A. L., Asthana, S., Johnson, S. C. 2010. NSAIDs may protect against age-related brain atrophy. Front. Aging Neurosci. 2:35.
University of Utah Health Services. 2010. Nobel winner ties mental illness to immune defect. e! Science News. http://esciencenews.com/articles/2010/05/27/nobel.winner.ties.mental.illness.immune.defect.
Chen, S. C., Tvrdik, P., Peden, E., Cho, S., Wu, S., Spangrude, G., Capecchi, M. R. 2010. Hematopoietic Origin of Pathological Grooming in Hoxb8 Mutant Mice. Cell. 141:775-785.
Carson, M. J., Bilousova, T. V., Puntambekar, S. S., Melchior, B., Doose, J. M., Ethell, I. M. 2007. A rose by any other name: the potential consequences of microglial heterogeneity during CNS health and disease. Neurotherapeutics. 4:571-579.
Colton, C. A., Gilbert, D. L. 1987. Production of superoxide anions by a CNS macrophage, the microglia. FEBS Letters. 223:284-288.
Enstrom, A. M., Lit, L., Onore, C. E., Gregg, J. P., Hansen, R. L., Pessah, I. N., Hertz-Picciotto, I., Van de Water, J. A., Pardo, C. A., Vargas, D. L., Zimmerman, A. W. 2005. Immunity, neuroglia and neuroinflammation in autism. Int. Rev. Psychiatry. 17:485-495.
Enstrom, A. M., Lit, L., Onore, C. E., Gregg, J. P., Hansen, R. L., Pessah, I. N., Hertz-Picciotto, I., Van de Water, J. A., Sharp, F. R., Ashwood, P. 2009. Altered gene expression and function of peripheral blood natural killer cells in children with autism. Brain Behav. Immun. 23:124-133.
Gehrmann, J., Matsumoto, Y., Kreutzberg, G. W. 1995. Microglia: intrinsic immuneffector cell of the brain. Brain Res. Rev. 20:269-287.
Hirschowitz, B. I. 1994. Nonsteroidal antiinflammatory drugs and the gastrointestinal tract. Gastroenterologist. 2:207-223.
Lue, L. F., Brachova, L., Civin, W. H., Rogers, J. 1996. Inflammation, A beta deposition, and neurofibrillary tangle formation as correlates of Alzheimer's disease neurodegeneration. J. Neuropathol. Exp. Neurol. 55:1083-1088.
Montalto, M., Gallo, A., Curigliano, V., D'Onofrio, F., Santoro, L., Covino, M., Dalvai, S., Gasbarrini, A, Gasbarrini, G. 2010. Clinical trial: the effects of a probiotic mixture on non-steroidal anti-inflammatory drug enteropathy—a randomized, double-blind, cross-over, placebo-controlled study. Aliment. Pharmacol. Ther. 32:209-214.
Owen, D. R., Matthews, P. M. 2011. Imaging brain microglial activation using positron emission tomography and translocator protein-specific radioligands. Int. Rev. Neurobiol. 101:19-39.
Pardo, C. A., Vargas, D. L., Zimmerman, A. W. 2005. Immunity, neuroglia and neuroinflammation in autism. Int. Rev. Psychiatry. 17:485-495.
Richardson, R. L., Kim, E. M., Gardiner, T., O'Hare, E. 2005. Chronic intracerebroventricular infusion of lipopolysaccharide: effects of ibuprofen treatment and behavioural and histopathological correlates. Behav. Pharmacol. 16:531-541.
Takabayashi, A., Kawai, Y., Iwata, S., Kanai, M., Denno, R., Kawada, K., Obama, K., Taki, Y. 2000. Nitric oxide induces a decrease in the mitochondrial membrane potential of peripheral blood lymphocytes, especially in natural killer cells. Antioxid. Redox Signal. 2:673-680.
Tremblay, M. E., Lowery, R. L., Majewska, A. K. 2010. Microglial interactions with synapses are modulated by visual experience. PLoS Biol. 8:e1000527.
Valko, M., Leibfritz, D., Moncol, J., Cronin, M. T., Mazur, M., Telser, J. 2007. Free radicals and antioxidants in normal physiological functions and human disease. Int. J. Biochem. Cell. Biol. 39:44-84.
Vandivier, R. W., Eidsath, A., Banks, S. M., Preas, H. L 2nd., Leighton, S. B., Godin, P. J., Suffredini, A. F., Danner, R. L. 1999. Down-regulation of nitric oxide production by ibuprofen in human volunteers. J. Pharmacol. Exp. Ther. 289:1398-1403.
Vargas, D. L., Nascimbene, C., Krishnan, C., Zimmerman, A. W., Pardo, C. A. 2005. Neuroglial activation and neuroinflammation in the brain of patients with autism. Ann. Neurol. 57:304.
Vojdani, A., Mumper, E., Granpeesheh, D., Mielke, L., Traver, D., Bock, K., Hirani, K., Neubrander, J., Woeller, K. N., O'Hara, N., Usman, A., Schneider, C., Hebroni, F., Berookhim, J., McCandless, J., 2008. Low natural killer cell cytotoxic activity in autism: the role of glutathione, IL-2 and IL-15. J. Neuroimmunol. 205:148-154.
Wilkinson, B. L., Cramer, P. E., Varvel, N. H., Reed-Geaghan, E., Jiang, Q., Szabo, A., Herrup, K., Lamb, B. T., Landreth, G. E. 2012. Ibuprofen attenuates oxidative damage through NOX2 inhibition in Alzheimer's disease. Neurobiology of Aging. 33:197.e31-197-e32 (available online Aug. 8, 2010).
Wood, P. L., 2003. Neuroinflammation: Mechanisms and Management. Humana Press.
Zimmerman, A. W., Jyonouchi, H., Comi, A. M., Connors, S. L., Milstein, S., Varsou, A., Heyes, M. P. 2005. Cerebrospinal fluid and serum markers of inflammation in autism. Pediatr. Neuro. 33:195-201.

1. A method of treating or ameliorating a condition with respect to a neurological disorder comprising microglial activation, the method comprising:
administering to an individual suffering from the condition:
a probiotic dosage comprising a therapeutically effective probiotic amount; and
an NSAID dosage comprising a therapeutically effective NSAID amount.

2. The method of claim 1, wherein:
the NSAID dosage is configured to modulate a microglial activation in the individual with respect to microglial release of neuro-inflammatory nitric oxide; and
the probiotic dosage is configured to at least one of prevent or restrict gastrointestinal lesions resulting from the NSAID dosage.

3. The method of claim 1, wherein:
the NSAID dosage is correlated to a weight of the individual; and
the probiotic dosage is of at least approximately 200 billion CFUs per day.

4. The method of claim 1, wherein:
the NSAID dosage comprises at least one of:
an ibuprofen dose of between approximately 30 mg/kg per day to approximately 90 mg/kg per day with respect to a weight of the individual; or
a dexibuprofen dose of between approximately 15 mg/kg per day to approximately 55 mg/kg per day with respect to the weight of the individual.

5. The method of claim 1, wherein:
the NSAID dosage comprises at least one of:
an ibuprofen dose of between approximately 60 mg/kg per day to approximately 90 mg/kg per day with respect to a weight of the individual; or
a dexibuprofen dose of between approximately 33 mg/kg per day to approximately 51 mg/kg per day with respect to the weight of the individual;
and
the probiotic dosage comprises between approximately 200 billion CFUs to approximately 400 billion CFUs.

6. The method of claim 1, wherein:
the probiotic dosage and the NSAID dosage are administered as part of a monthly treatment regimen comprising approximately 30 days;
the probiotic dosage is configured to be administered daily throughout the monthly treatment regimen; and
the NSAID dosage is configured to be administered daily throughout a subset of days of the monthly treatment regimen.

7. The method of claim 6, wherein:
the subset of days comprises at least approximately 10 consecutive days.

8. The method of claim 1, wherein:
administering the probiotic dosage to the individual comprises:
administering a first probiotic dose at least approximately 30 minutes prior to a first daily meal of the individual; and
administering a second probiotic dose at least approximately 30 minutes after a last daily meal of the individual; and
each of the first and second probiotic doses comprises approximately half of the probiotic dosage.

9. The method of claim 1, wherein:
administering the NSAID dosage to the individual comprises:
administering a first NSAID dose along with a first daily meal of the individual;
administering a second daily NSAID dose along with a second daily meal of the individual; and
administering a third daily NSAID dose along with a third daily meal of the individual.

10. The method of claim 1, wherein:
the condition comprises at least one of an Alzheimer's condition, an amyotrophic lateral sclerosis (ALS) condition, a Parkinson's disease condition, a cardiovascular disease condition, a stroke condition, a brain cancer condition, a schizophrenia condition, a depression condition, an obsessive-compulsive disorder (OCD) condition, a Huntington's disease condition, a sleep disorders condition, a Rett's syndrome condition, a adrenoleukodystrophy condition, or a Tourette's syndrome condition.

11. The method of claim 1, further comprising:
administering to the individual suffering from the condition:
an antiviral dosage comprising a therapeutically effective amount of antiviral agents against one or more microglial-activation-inducing viruses.

12. The method of claim 11, wherein:
the valacyclovir dosage comprises at least one of:
an adult valacyclovir dosage of at least approximately 3 grams per day; or
a child valacyclovir dosage of at least approximately 21.4 mg per day;
the famciclovir dosage comprises at least one of:
an adult famciclovir dosage of at least approximately 1.5 grams per day; or
a child famciclovir dosage of at least approximately 10.7 mg per day;
and
the acyclovir dosage is of at least approximately 36.3 mg/lb per day.

13. The method of claim 1, further comprising:
administering to the individual suffering from the condition:
an antifungal dosage comprising a therapeutically effective amount of antifungal agents against one or more microglial-activation-inducing fungal infections;
wherein the antifungal dosage comprises at least one of:
a ketoconazole dosage of at least approximately 4 mg/kg, up to 200 mg per day; or
a fluconazole dosage of at least approximately 4 mg/kg, up to 200 mg per day.

14. The method of claim 1, further comprising:
administering to the individual suffering from the condition:
an immunomodulator dosage comprising a therapeutically effective amount of immunomodulator agents to modulate cell-mediated immunity via T-lymphocytes and NK cell cytotoxicity.

15. The method of claim 14, wherein:
if the individual weighs up to 20 kg, the immunomodulator dosage comprises:
a child inosine pranobex dosage of at least approximately 50 mg/kg, up to 20 kg;
Or
if the individual weighs over 20 kg, the immunomodulator dosage comprises one of:
an adult inosine pranobex dosage of at least approximately 50 mg/kg per day, up to 3 grams;
an adult inosine pranobex dosage of at least approximately 1 gram three times per day; or
an adult inosine pranobex dosage of at least approximately 1 gram four times per day.

16. The method of claim 1, further comprising:
administering to the individual suffering from the condition:
a glutathione dosage comprising a therapeutically effective amount of glutathione to reduce oxidative stress in the brain caused by elevated levels of nitric oxide released by chronically active microglia;
wherein the glutathione dosage is of at least approximately 250 mg per day.

17. The method of claim 1, further comprising:
administering to the individual suffering from the condition:
an LDN (low-dose naltrexone) dosage comprising a therapeutically effective amount of naltrexone to increase NK cell activity for fighting microglia-activating infections;
wherein the LDN dosage is of at least approximately 3 mg per day.

18. In combination, for treating or ameliorating a neurological disorder comprising microglial activation in an individual:
a probiotic dosage of a therapeutically effective probiotic amount; and
an NSAID dosage of a therapeutically effective NSAID amount.

19. The combination of claim 18, comprising:
a pharmaceutical composition comprising:
at least a portion of the probiotic dosage of the therapeutically effective probiotic amount; and
at least a portion of the NSAID dosage of the therapeutically effective NSAID amount.

20. The combination of claim 19, wherein:
the pharmaceutical composition comprises:
a pharmaceutically acceptable carrier vehicle in at least one of a solid form or a liquid solution form and configured to disperse the portion of the probiotic dosage and the portion of the NSAID dosage.

21. The combination of claim 18, wherein:
the probiotic dosage and the NSAID dosage are separately administrable to the individual.

22. The combination of claim 18, wherein:
the NSAID dosage is configured to inhibit the microglial activation in the individual; and
the probiotic dosage is configured to at least one of prevent or restrict gastrointestinal lesions resulting from the therapeutically effective NSAID dose.

23. The combination of claim 18, wherein:
the NSAID dosage comprises at least one of:
an ibuprofen dose of between approximately 60 mg/kg per day to approximately 90 mg/kg per day with respect to a weight of the individual; or
a dexibuprofen dose of between approximately 33 mg/kg per day to approximately 51 mg/kg per day with respect to the weight of the individual;
and
the probiotic dosage comprises at least approximately 200 billion CFUs.

24. The combination of claim 18, further comprising at least one of:
an antiviral dosage of a therapeutically effective antiviral amount against one or more microglial-activation-inducing viruses;
an antifungal dosage of a therapeutically effective antifungal amount against one or more microglial-activation-inducing fungal infections;
an immunomodulator dosage of a therapeutically effective immunomodulator amount to modulate cell-mediated immunity via T-lymphocytes and NK cell cytotoxicity regulation;
a glutathione dosage of a therapeutically effective glutathione amount to reduce oxidative stress in the brain caused by elevated levels of nitric oxide released by chronically active microglia; or
an LDN dosage of a therapeutically effective LDN amount to increase NK cell activity for fighting microglia-activating infections.

25. The combination of claim 19, wherein:
the therapeutically effective probiotic amount of the probiotic dosage and the therapeutically effective NSAID amount of the NSAID dosage are at least one of marketed, distributed, or sold together.

26. A method of treating or ameliorating a neurological disorder comprising microglial activation, the method comprising:
administering to an individual suffering from the condition:
a probiotic dosage comprising a therapeutically effective probiotic amount; and
an NSAID dosage comprising a therapeutically effective NSAID amount;
wherein:
the NSAID dosage is configured to inhibit a microglial activation in the individual;
the probiotic dosage is configured to at least one of prevent or restrict gastrointestinal lesions resulting from the NSAID dosage;
the probiotic dosage is of between approximately 200 billion CFUs to approximately 400 billion CFUs per day;
and
the NSAID dosage comprises at least one of:
an ibuprofen dose of between approximately 60 mg/kg per day to approximately 90 mg/kg per day with respect to a weight of the individual; or
a dexibuprofen dose of between approximately 33 mg/kg per day to approximately 51 mg/kg per day with respect to the weight of the individual.
